(19) United States
(12) Patent Application Publication
(10) Pub. No.: US 2013/0079290 A1
(43) Pub. Date: Mar. 28, 2013

(54) S1P ANTAGONISTS AS ADJUNCT OCULAR HYPOTENSIVES

(71) Applicants: David F. Woodward, Lake Forest, CA (US); Todd M. Heidelbaugh, Fountain Valley, CA (US); W. Daniel Stamer, Durham, NC (US)

(72) Inventors: David F. Woodward, Lake Forest, CA (US); Todd M. Heidelbaugh, Fountain Valley, CA (US); W. Daniel Stamer, Durham, NC (US)

(73) Assignee: ALLERGAN, INC., Irvine, CA (US)

(21) Appl. No.: 13/430,340

(22) Filed: Mar. 26, 2012

Related U.S. Application Data
(60) Provisional application No. 61/467,690, filed on Mar. 25, 2011.

(51) Int. Cl. A61K 45/06; A61K 31/5575; A61K 31/4178; A61K 38/17

(52) U.S. Cl. CPC A61K 45/06; A61K 38/177; A61K 31/5575; A61K 31/4178
USPC 514/20.6

(57) ABSTRACT

The invention provides compositions and methods for further reducing IOP in a subject who has already achieved maximal IOP reduction using known IOP lowering agents. The activity of an ocular hypotensive treatment regimen may be increased by adding an S1P antagonist to prevent S1P mediated reversal as a result of decreased aqueous humor outflow.

### RELATED APPLICATION

This application claims the benefit of U.S. Provisional Application Ser. No. 61/467,690, filed Mar. 25, 2011, the disclosure of which is hereby incorporated in its entirety herein by reference

### FIELD OF THE INVENTION

This invention is directed to SIP antagonists in combination with known IOP lowering drugs for lowering intraocular pressure.

### BACKGROUND OF THE INVENTION

It is well-known that efforts to profoundly reduce intraocular pressure (IOP) in ocular hypertensive patients by pharmacological interventions rarely meet expectations. For example, the ocular hypotensive activity of once daily bimatoprost (0.03%) is not increased by higher concentrations or by increased dosing frequency; ocular hypotensive efficacy is actually decreased. Further, ocular hypotensive drug combinations typically fall short of theoretical expectations. There appears to be a “glass floor” that prevents drug mediated ocular hypotension from descending below a certain level.

Sphingosine-1-phosphate (S1P) is unique in that it pharmacologically acts as a naturally occurring “local hormone” that actually decreases aqueous humor outflow (Stamer et al., 2009). In contrast, all other pharmacologically active substances increase aqueous outflow, with a resultant decrease in IOP. A potential physiological role for S1P is in preventing profound popular hypotony that may occur in disease states, for example uveitis. Thus, S1P prevents IOP from falling below a certain level by decreasing aqueous humor outflow.

### SUMMARY OF THE INVENTION

The invention provides compositions and methods for further reducing IOP in a subject who has already achieved maximal IOP reduction using known IOP lowering agents. The activity of an ocular hypotensive treatment regimen may be increased by adding an S1P antagonist to prevent S1P mediated reversal as a result of decreased aqueous humor outflow.

Thus, in one embodiment of the invention, there are provided compositions including an S1P antagonist and at least one compound selected from the group consisting of β-blockers, adrenergic agonists, non-selective adrenergic agonists, α₂-selective adrenergic agonists, carbonic anhydrase inhibitors, cholinergic agonists, direct acting cholinergic agonists, chlolinesterase inhibitors, glutamate antagonists, Ca²⁺ channel blockers, prostamides, prostaglandins, cannabinoids, muscarinic agents, and combinations thereof.

In another embodiment, there are provided methods for lowering IOP in a subject in need thereof. Such methods can be performed, for example, by administering to a subject a therapeutically effective amount of the composition including an S1P antagonist and at least one compound selected from the group consisting of β-blockers, adrenergic agonists, non-selective adrenergic agonists, α₂-selective adrenergic agonists, carbonic anhydrase inhibitors, cholinergic agonists, direct acting cholinergic agonists, chlolinesterase inhibitors, glutamate antagonists, Ca²⁺ channel blockers, prostamides, prostaglandins, cannabinoids, muscarinic agents, and combinations thereof.

In another embodiment, there are provided methods for further reducing IOP in a subject already being treated with a composition including at least one compound selected from the group consisting of β-blockers, adrenergic agonists, non-selective adrenergic agonists, α₂-selective adrenergic agonists, carbonic anhydrase inhibitors, cholinergic agonists, direct acting cholinergic agonists, chlolinesterase inhibitors, glutamate antagonists, Ca²⁺ channel blockers, prostamides, prostaglandins, cannabinoids, muscarinic agents, and combinations thereof; such methods can be performed, for example, by administering to the subject in need thereof a therapeutically effective amount of a composition including an S1P antagonist and at least one compound selected from the group consisting of β-blockers, adrenergic agonists, non-selective adrenergic agonists, α₂-selective adrenergic agonists, carbonic anhydrase inhibitors, cholinergic agonists, direct acting cholinergic agonists, chlolinesterase inhibitors, glutamate antagonists, Ca²⁺ channel blockers, prostamides, prostaglandins, cannabinoids, muscarinic agents, and combinations thereof.

### DETAILED DESCRIPTION OF THE INVENTION

It is to be understood that both the foregoing general description and the following detailed description are exemplary and explanatory only and are not restrictive of the invention claimed. As used herein, the use of the singular includes the plural unless specifically stated otherwise. As used herein, “or” means “and/or” unless stated otherwise. Furthermore, use of the term “including” as well as other forms, such as “includes,” and “included,” is not limiting. The section headings used herein are for organizational purposes only and are not to be construed as limiting the subject matter described.

As used herein, hydrocarbyl consists of carbon and hydrogen, wherein each carbon has 4 covalent bonds and each hydrogen has a single bond to a carbon atom. “Hydrocarbyl fragments” has the same meaning as “hydrocarbyl,” but is merely used for convenience for counting purposes. For example, one or more hydrocarbyl fragments means, 1, 2, or more distinct parts that each consists of hydrocarbyl, which may be interrupted by another moiety. For example, a functional group may be attached to 2 distinct hydrocarbyl fragments.

Hydrocarbyl includes alkyl, alkenyl, alkynyl, aryl containing only hydrogen and carbon, and combinations thereof. Hydrocarbyl may be linear, branched, cyclic (aromatic or non-aromatic), or combinations thereof, which can be further substituted.

Alkyl is a hydrocarbyl having no double bonds. Examples include methyl, ethyl, propyl isomers, butyl isomers, pentyl isomers, hexyl isomers, cyclopropyl, cyclobutyl, cyclopentyl, cyclohexyl, etc.

Alkenyl is a hydrocarbyl having one or more double bonds. Examples include ethenyl, propenyl, butenyl isomers, pentenyl isomers, hexenyl isomers, cyclopentenyl, cyclohexenyl, etc.

Alkynyl is a hydrocarbyl having one or more triple bonds. Examples include ethynyl, propynyl, butynyl isomers, pentynyl isomers, hexynyl isomers, cyclopentynyl, cyclohexynyl, etc.

Aryl is a substituted or unsubstituted aromatic ring or ring system. It can be hydrocarbon-aryl or heteroaryl. Examples of hydrocarbon-aryl include substituted and unsubstituted phenyl, naphthyl, and biphenyl. Such aryl group can be bonded to other moieties within the molecule at any position.

Each hydrogen atom has one covalent bond to carbon (C), nitrogen (N), oxygen (O), or sulfur (S).

Halo or halo atoms are fluorine (F), chlorine (Cl), bromine (Br), and iodine (I). Each halo atom forms a single bond to a carbon atom. Halohydrocarbyl is a hydrocarbyl having one or more F, Cl, Br, or I as substituents.

Heterohydrocarbyl refers to a hydrocarbyl as defined above with at least one non-carbon atom(s) presented at the backbone, including but not limiting to, oxygen (O), sulfur (S), nitrogen (N), phosphor (P), and halo atoms. Heterohydrocarbyl may be linear, branched, cyclic (aromatic or non-aromatic), or combinations thereof, which can be further substituted.

Examples of heterohydrocarbyl include: —R¹⁰⁻G¹-R¹¹, —R¹⁰—H1, -G¹-R¹⁰, -G¹-R¹⁰—H1, G¹-R¹⁰-G², and G¹-R¹⁰-G²-R¹¹, wherein R¹⁰ and R¹¹ are independently hydrocarbyl or hydrogen (provided that hydrogen is attached to only one C, N, O, or S atom), G¹ and G² are independently functional groups, and H1 is halo.

The invention provides aqueous ophthalmic compositions including an S1P antagonist and at least one compound selected from the group consisting of β-blockers, adrenergic agonists, non-selective adrenergic agonists, α₂-selective adrenergic agonists, carbonic anhydrase inhibitors, cholinergic agonists, direct acting cholinergic agonists, chlolinesterase inhibitors, glutamate antagonists, Ca²⁺ channel blockers, prostamides, prostaglandins, cannabinoids, muscarinic agents, and combinations thereof.

S1P antagonists contemplated for use in the practice of the invention include, but are not limited to, S1P2 and S1P3 antagonists. In certain embodiments, the S1P antagonist is either a selective or non-selective antagonist.

In some embodiments, the S1P antagonists are compounds having the structure :

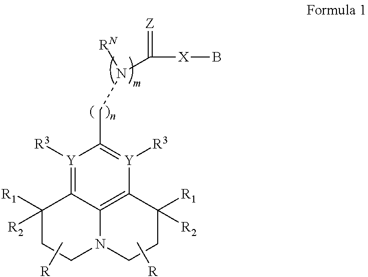

wherein, when referring to Formula 1, m is an integer of 0, or 1; n is an integer of 0, 1, 2, or 3; each Y is independently carbon (C) or nitrogen (N); Z and X are each independently selected from the group of oxygen (O), sulfur (S), and amine moiety NR^{N}; B is selected from the group consisting of hydrogen, hydrocarbyl, heterohydrocarbyl, substituted or unsubstituted aryl, halohydrocarbyl, hydroxyl, alkoxyl, hydroxyalkyl, alkylcarbonyl, carbonylalkyl, formyl, oxycarbonyl, aminocarbonyl, alkyl carboxyl, alkyl amide, amino, alkylamino, cyano and X-B together being a heterocyclic ring or ring system; R and R³ are each independently selected from the group consisting of hydrogen, hydrocarbyl, heterohydrocarbyl, substituted or unsubstituted aryl, halo, halohydrocarbyl, hydroxyl, alkoxyl, hydroxyalkyl, alkylcarbonyl, carbonylalkyl, formyl, oxycarbonyl, aminocarbonyl, alkyl carboxyl, alkyl amide, amino, alkylamino, and cyano; each R¹ is independently selected from the group consisting of hydrogen, hydrocarbyl, heterohydrocarbyl, substituted or unsubstituted aryl, halo, halohydrocarbyl, hydroxyl, alkoxyl, hydroxyalkyl, alkylcarbonyl, formyl, oxycarbonyl, aminocarbonyl, aminocarbonxyl, alkylcarboxyl, alkyl amide, amino, alkylamino, and cyano; each R² is independently selected from the group consisting of hydrocarbyl, heterohydrocarbyl, substituted or unsubstituted aryl, halo, halohydrocarbyl, hydroxyl, alkoxyl, hydroxyalkyl, alkylcarbonyl, formyl, oxo, oxycarbonyl, carboxyl, alkyl carboxylate, alkyl amide, aminocarbonyl, amino, alkylamino, and cyano; each R^{N} is independently selected from the group consisting of hydrogen, hydrocarbyl, heterohydrocarbyl, substituted or unsubstituted aryl, halohydrocarbyl, hydroxyl, alkoxyl, hydroxyalkyl, alkylcarbonyl, carbonylalkyl, formyl, oxycarbonyl, aminocarbonyl, alkyl carboxyl, alkyl amide, amino, alkylamino, and cyano; including their alternate solid forms, tautomers, stereoisomers, enantiomers, diastereomers, prodrugs, and pharmaceutically acceptable salts, hydrates and solvates; and provided that when Y is carbon, and Z and X are both oxygen, R² is not oxo, or R¹ and R² are not both phenyl or both methyl at the same time.

In some embodiments of the invention, the S 1P antagonists are compounds having the structure

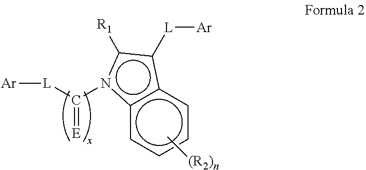

wherein, when referring to Formula 2

each Ar is independently substituted or unsubstituted aryl or heteroaryl;

each L is independently alkylene, alkenylene, oxyalkylene, oxyalkenylene, aminoalkylene, or aminoalkenylene;

R₁ is lower alkyl, alkylacyl or hydroxyalkyl;

each R₂ is independently H, lower alkyl, halide, trifluoromethyl, lower alkenyl, lower alkynyl, cycloalkyl, —CN, —CH₂CN, —CH₂SR₃, —CH₂N(R₃)₂, —CH₂OR₃, —CH═NOR₃, —OR₃, —SR₃, —N(R₃)₂, —C(O)R₄, heterocycle, substituted heterocycle, aryl, substituted aryl, heteroaryl, substituted heteroaryl; or

R₂ is

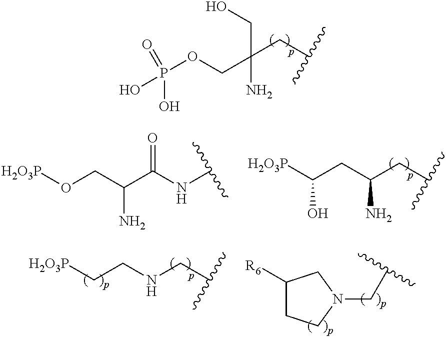

wherein R₅ is —CO₂H or PO₃H₂ and p is 0-2; or

when n is 2, each R₂ taken together with carbon atoms to which each R₂ is attached forms an aryl, substituted aryl, heteroaryl, substituted heteroaryl, cycloalkyl or substituted cycloalkyl;

each R₃ is independently H, lower alkyl, cycloalkyl, allyl, phenyl, substituted phenyl, heteroaryl, or substituted heteroaryl;

each R₄ is independently H, lower alkyl, cycloalkyl, alkoxy, alkyamino, dialkylamino, phenyl, substituted phenyl, heteroaryl, substituted heteroaryl, or trifluoromethyl;

E is O or S;

x is 0 or 1; and

n is 0-5;

or pharmaceutically acceptable salts thereof.

In still another embodiment of the invention, the S1P antagonists are compounds having the structure:

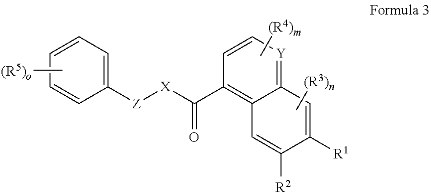

wherein, when referring to Formula 3:

n is 0, 1, or 2;

m is 1 or 2;

o is from 0 to 5;

one of R¹ and R² has a formula C₁₋₉H₀₋₂₃N₀₋₄O₀₋₄S₀₋₄F₀₋₆Cl₀₋₄Br₀₋₄I₀₋₄, and is selected from: a substituted or unsubstituted heterocycle having 5 or 6 atoms in the ring; and Cy, —S-Cy, —NH-Cy, and —O-Cy, wherein Cy is a substituted or unsubstituted carbocycle or a substituted or unsubstituted heterocycle;

one of R¹ and R² is hydrogen or a substituent having a formula C₀₋₁₂H₀₋₂₆N₀₋₂O₀₋₄S₀₋₁P₀₋₁F₀₋₆Cl₀₋₁Br₀₋₁I₀₋₁;

each R³, R⁴, and R⁵ are independently a substituent having a formula C₀₋₁₂H₀₋₂₆N₀₋₂O₀₋₄S₀₋₁P₀₋₁F₀₋₆Cl₀₋₁Br₀₋₁I₀₋₁;

Y is N or C—H or C—R⁴;

X is O, S, NH, N-alkyl having from 1 to 4 carbon atoms, or a bond; and

Z is hydrocarbyl having a formula C₁₋₈H₄₋₁₇.

In another embodiment of the invention, the S1P antagonists are compounds having the structure:

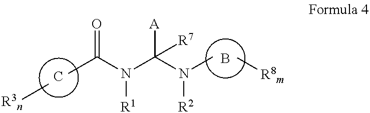

wherein, when referring to Formula 4, R¹ and R² are each independently selected from H and C₁-C₄ alkyl;

C is a phenyl, aryl or heteroaryl having the structure

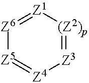

wherein the dashed line represents the presence or absence of a bond, and wherein p is 0-1, and Z¹-Z⁶ are each independently selected from C, N, O or S;

R³ and R⁸ are each independently selected from H, C₁-C₆ straight or branched chain alkyl, alkenyl, or alkynl, alkoxy (such as O(C₁-C₆)), —OH, halogen, —NR⁴₂, —CN, —CO₂R⁴, —C(O)NR⁴R⁵, —CH₂OH, —CF₃, —OCHF₂, —OCF₃, —NO₂, alkylamino, or alkylcarboxyl;

m is 0-5;

n is 0-5;

R⁴ and R⁵ are each independently selected from H, C₁-C₆, branched or unbranched alkyl, alkenyl, or alkynl, C₃-C₆ saturated or unsaturated cyclic hydrocarbon, aryl, heteroaryl, haloalkyl, hydroxyl, alkoxyl, hydroxyalkyl, alkylcarbonyl, formyl, oxycarbonyl, carboxyl, alkyl carboxylate, alkylamide, alkylamino aminocarbonyl, or amino;

A is CR⁶₃, CXR⁶₂, CX₂R⁶, CX₃, COQ¹, SOQ¹, SO₂Q¹, CSQ¹, phenyl, substituted phenyl, heterocylic, heteroaromatic, cycloalkyl, cycloalkenyl sulfonyl, sulfone, sulfonamide, sulfoxide, ester, or thiocarbonyl;

X is a halogen;

R⁶ is H, C₁-C₆ straight or branched chain alkyl, alkenyl, or alkynyl, haloalkyl, perfluorinated alkyl, partially fluorinated alkyl, perhalogenated alkyl, partially halogenated alkyl, phenyl, substituted phenyl, heteroaryl, cyano, ketyl, and the like;

Q¹ is an aryl or heteroaryl variably substituted with (R³)ₙ, a phenyl, heteroaromatic or cycloalkyl, cycloalkenyl, or partially saturated or saturated heterocyclic ring a bicyclic compound, NR⁴R⁵;

R⁷ is H, C₁-C₆ branched or unbranched alkyl, alkenyl, or alkynl, haloalkyl, aryl, heteroaryl, perfluorinated alkyl and partially fluorinated alkyl, phenyl, cyano, ketyl, CF₃, substituted aryl or heteroaryl or spirocyclic compounds; and

B is phenyl, aryl, heteroaromatic or cycloalkyl, cycloalkenyl, or partially saturated or saturated heterocyclic ring, or a bicyclic compound, with the proviso that when A is CX₃, B is not phenyl.

In another embodiment of the invention, the S1P antagonists are compounds having the structure:

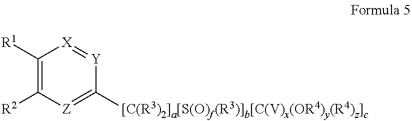

wherein, when referring to Formula 5:

X is selected from the group consisting of CR³, N and NO;

Y is selected from the group consisting of CR³, N and NO;

Z is selected from the group consisting of CR³, N and NO;

and at least one of X, Y and Z is N or NO;

V is O or NOR⁴

R¹ is an aryl group;

R² is an aryl group;

R³ is selected from the group consisting of H and alkyl; and 2 of said R³ groups may together form a cyclic alkyl ring having from 3 to 6 carbon atoms;

R⁴ is selected from the group consisting of H and alkyl;

a is 0 or an integer of from 1 to 6;

b is 0 or 1;

c is 0 or 1;

f is 0 or an integer of 1 or 2;

x is 0 or 1;

y is 0 or an integer of from 1 to 3; and

z is 0 or an integer of from 1 to 3.

In another embodiment of the invention, the S1P antagonists are compounds having the structure:

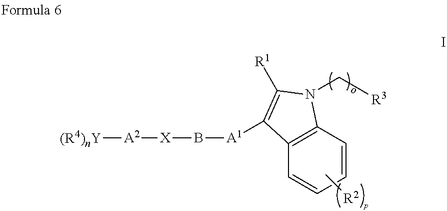

wherein, when referring to Formula 6:

A¹ and A² are independently selected from the group consisting of (CH₂)m where m is 0 or an integer of from 1 to 6, lower branched chain alkyl having 2 to 6 carbons, cycloalkyl having 3 to 6 carbons, alkenyl having 2 to 6 carbons and 1 or 2 double bonds, alkynyl having 2 to 6 carbons and having 1 or 2 triple bonds, NR⁵, O and S;

B is selected from the group consisting of (CH₂)n, where n is 0 or an integer of from 1 to 6, lower branched chain alkyl having 2 to 6 carbons, cycloalkyl having 3 to 6 carbons, alkenyl having 2 to 6 carbons and 1 or 2 double bonds, alkynyl having 2 to 6 carbons and having 1 or 2 triple bonds, C═C(R⁵)₂, C═O, C═S, R⁵C═NR⁵, R⁵C═CR⁵, C═NOR⁵, CR⁵OR⁵, C(OR⁵)₂, CR⁵N(R⁵)₂, C(N(R⁵)₂)₂, CR⁵SR⁵, C(SR⁵)₂,_SO, SO₂, and heterocyclic aryl comprising from 2 to 14 carbon atoms and from 1 to 3 heteroatoms selected from the group consisting of nitrogen, oxygen and sulfur;

X is selected from the group consisting of (CH₂)r, where r is 0 or an integer of from 1 to 6, lower branched chain alkyl having 2 to 6 carbons, cycloalkyl having 3 to 6 carbons, alkenyl having 2 to 6 carbons and 1 or 2 double bonds, alkynyl having 2 to 6 carbons and having 1 or 2 triple bonds, NR⁵, O and S;

provided that when m is 0 and B is C═O then X is not NR⁵, O or S;

Y is R⁶, or a carbocyclic aryl group comprising from 6 to 14 carbon atoms or a heterocyclic aryl group comprising from 2 to 14 carbon atoms and from 1 to 3 heteroatoms selected from the group consisting of nitrogen, oxygen and sulfur, and, preferably, Y is a phenyl group, or heterocyclic aryl group selected from the group consisting of pyridyl, thienyl, furyl, pyradizinyl, pyrimidinyl, pyrazinyl, thiazolyl, oxazolyl, and imidazolyl;

o is 0 or an integer of from 1 to 3;

p is 0 or an integer of from 1 to 4;

R¹, R², R³, R⁴ are independently selected from the group consisting of hydrogen, straight or branched chain alkyl having 1 to 12 carbons, cycloalkyl having 3 to 6 carbons, alkenyl having 2 to 6 carbons and 1 or 2 double bonds, alkynyl having 2 to 6 carbons and 1 or 2 triple bonds, aryl, preferably a carbocyclic aryl group having from 6 to 14 carbon atoms or a heterocyclic aryl group having from 2 to 14 carbon atoms and from 1 to 3 heteroatoms selected from the group consisting of nitrogen, oxygen and sulfur, halo, e.g. fluoro or chloro, C₁ to C₁₂ haloalkyl, e.g. trifluoromethyl, hydroxyl, C₁ to C₁₂ alkoxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl, sulfonyl,

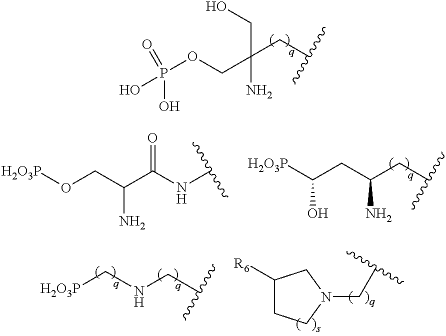

wherein R is CO₂H or PO₃H₂ and q is 0 or an integer of 1 to 5 and s is 0 or an integer from 1 to 3;

R⁵ is selected from the group consisting of hydrogen, straight or branched chain alkyl having 1 to 12 carbons, cycloalkyl having 3 to 6 carbons, alkenyl having 2 to 6 carbons and 1 or 2 double bonds, alkynyl having 2 to 6 carbons and 1 or 2 triple bonds, aryl, preferably a carbocyclic aryl group having from 6 to 14 carbon atoms or a heterocyclic aryl group having from 2 to 14 carbon atoms and from 1 to 3 heteroatoms selected from the group consisting of nitrogen, oxygen and sulfur, halo, e.g. fluoro or chloro, C₁ to C₁₂ haloalkyl, hydroxyl, C₁ to C₁₂ alkoxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl, and sulfonyl; and

R⁶ is selected from the group consisting of straight or branched chain alkyl, having 1 to 12 carbons, cycloalkyl having 3 to 6 carbons, alkenyl having 2 to 6 carbons and 1 or 2 double bonds and alkynyl having 2 to 6 carbons and 1 or 2 triple bonds.

In another embodiment of the invention, the S1P antagonists are compounds having the structure:

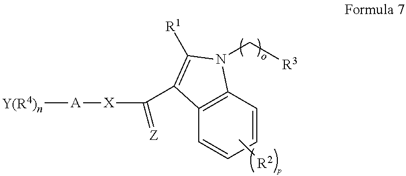

wherein, when referring to Formula 7:

X is NR⁵, O, S;

Z is O or S;

n is 0 or an integer of from 1 to 5, e.g. 1 to 4;

o is 0 or an integer of from 1 to 3;

p is 0 or an integer of from 1 to 4, e.g. 1 to 3;

A is (C(R⁵)₂)m, wherein

m is 0 or an integer of from 1 to 6;

R⁵ is selected from the group consisting of hydrogen, straight or branched chain alkyl having 1 to 12 carbons, cycloalkyl having 3 to 6 carbons, alkenyl having 2 to 6 carbons and 1 or 2 double bonds, alkynyl having 2 to 6 carbons and 1 or 2 triple bonds, aryl (as defined below), halo, C₁ to C₁₂ haloalkyl, hydroxyl, C₁ to C₁₂ alkoxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl, or sulfonyl groups;

Y is a carbocyclic aryl or heterocyclic aryl group wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur, and wherein said aryl may be bonded to A at any position;

R¹, R², R³, R⁴ are selected from the group consisting of hydrogen, straight or branched chain alkyl having 1 to 12 carbons, cycloalkyl having 3 to 6 carbons, alkenyl having 2 to 6 carbons and 1 or 2 double bonds, alkynyl having 2 to 6 carbons and 1 or 2 triple bonds, aryl (as defined below), halo, C₁ to C₁₂ haloalkyl, hydroxyl, C₁ to C₁₂ alkoxy, C₃ to C₂₀ arylalkyloxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl, or sulfonyl groups, or a group selected from the group consisting of:

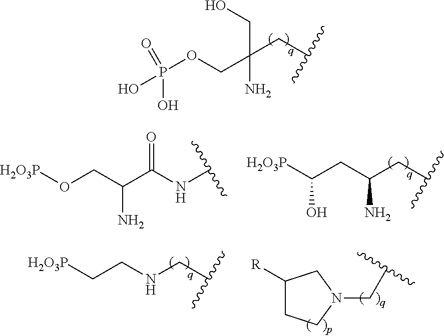

wherein R is CO₂H or PO₃H₂, p is an integer of 1 or 2 and q is 0 or an integer of 1 to 5.

The compositions and methods for use thereof described herein include S1P antagonists in combination with at least one compound selected from the group consisting of β-blockers, adrenergic agonists, non-selective adrenergic agonists, α₂-selective adrenergic agonists, carbonic anhydrase inhibitors, cholinergic agonists, direct acting cholinergic agonists, chlolinesterase inhibitors, glutamate antagonists, Ca²⁺ channel blockers, prostamides, prostaglandins, cannabinoids, muscarinic agents, and combinations thereof.

Examples of these compounds include, but are not limited to:

β-Blockers (or (β-adrenergic antagonists) including carteolol, levobunolol, metiparanolol, timolol hemihydrate, timolol maleate, β1-selective antagonists such as betaxolol, and the like, or pharmaceutically acceptable salts or prodrugs thereof;

Adrenergic Agonists including

non-selective adrenergic agonists such as epinephrine borate, epinephrine hydrochloride, and dipivefrin, and the like, or pharmaceutically acceptable salts or prodrugs thereof; and

α₂-selective adrenergic agonists such as apraclonidine, brimonidine, and the like, or pharmaceutically acceptable salts or prodrugs thereof;

Carbonic Anhydrase Inhibitors including acetazolamide, dichlorphenamide, methazolamide, brinzolamide, dorzolamide, and the like, or pharmaceutically acceptable salts or prodrugs thereof;

Cholinergic Agonists including

direct acting cholinergic agonists such as carbachol, pilocarpine hydrochloride, pilocarbine nitrate, pilocarpine, and the like, or pharmaceutically acceptable salts or prodrugs thereof;

chlolinesterase inhibitors such as demecarium, echothiophate, physostigmine, and the like, or pharmaceutically acceptable salts or prodrugs thereof;

Glutamate Antagonists and other neuroprotective agents such as Ca²⁺ channel blockers such as memantine, amantadine, rimantadine, nitroglycerin, dextrophan, detromethorphan, CGS-19755, dihydropyridines, verapamil, emopamil, benzothiazepines, bepridil, diphenylbutylpiperidines, diphenylpiperazines, HOE 166 and related drugs, fluspirilene, eliprodil, ifenprodil, CP-101,606, tibalosine, 2309BT, and 840S, flunarizine, nicardipine, nifedimpine, nimodipine, barnidipine, verapamil, lidoflazine, prenylamine lactate, amiloride, and the like, or pharmaceutically acceptable salts or prodrugs thereof;

Prostamides such as bimatoprost, or pharmaceutically acceptable salts or prodrugs thereof; and

Prostaglandins including travoprost, UFO-21, chloprostenol, fluprostenol, 13,14-dihydrochloprostenol, isopropyl unoprostone, latanoprost, tafluprost, and the like.

Cannabinoids including CB1 agonists such as WIN-55212-2 and CP-55940 and the like, or pharmaceutically acceptable salts or prodrugs thereof.

steroids including triamcinolone, dexamethasone, fluocinolone, and the like.

An effective amount of the compositions disclosed herein is an amount useful to observe a therapeutic effect as compared to a placebo formulation that, except for the absence of composition disclosed herein, is otherwise identical to the formulation. The amount of the composition to administer depends on factors such as the intended therapeutic effects, the specific subject in need thereof, the severity and nature of the subject's condition, the manner of administration, the potency and pharmacodynamics of the particular compound, and the judgment of the prescribing physician. In some embodiments of the invention, the therapeutically active agents of the composition are present at a concentration of 0.01 to 0.12% w/v. In other embodiments, the therapeutically active agents of the composition are present at a concentration of 0.05 to 0.1% (w/v). In certain embodiments, the therapeutically active agents of the composition are present at a concentration of 0.05%, 0.075%, or 0.01% (w/v).

A liquid which is ophthalmically acceptable is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants. The formulations or compositions of the present invention maybe in the form of solutions, emulsions, reverse-emulsions, micro-emulsions or delivered by a bioerodable or non-bioerodable device or ocular implant.

As is known in the art, buffers are commonly used to adjust the pH to a desirable range for ophthalmic use. Generally, a pH of around 6-8 is desired, however, this may need to be adjusted due to considerations such as the stability or solubility of the therapeutically active agent or other excipients. In some embodiments of the invention, the buffer maintains the pH between 6.5 and 7.5. In other embodiments, the buffer maintains the pH between 7.0 and 7.4. Many buffers including salts of inorganic acids such as phosphate, borate, and sulfate are known. In some embodiments of the invention a phosphate/phosphoric acid buffer is used in the formulations described herein. The term “phosphate/phosphoric acid” refers to any combination of phosphoric acid and one or more of the conjugate bases such that the pH is adjusted to the desired range. In other embodiments borate/boric acid buffer is used. In still other embodiments a citrate/citric acid buffer is used in the formulations described herein. In certain embodiments a combination of phosphate/phosphoric acid buffer and citrate/citric acid buffer is used in the formulations described herein.

In ophthalmically acceptable liquids tonicity agents often are used to adjust the composition of the formulation to the desired isotonic range. Tonicity agents are well known in the art and some examples include glycerin, mannitol, sorbitol, sodium chloride, and other electrolytes. In some embodiments of the invention, the tonicity agent is present in the formulation at a concentration of 1.20 to 1.25% w/v. In one embodiment, the tonicity agent is present at a concentration of 1.22% w/v.

A surfactant may be used for assisting in dissolving an excipient or a therapeutically active agent, dispersing a solid or liquid in a composition, enhancing wetting, modifying drop size, or a number of other purposes. Useful surfactants, include, but are not limited to sorbitan esters, Polysorbate 20, Polysorbate 40, Polysorbate 60, Polysorbate 80, stearates, glyceryl stearate, isopropyl stearate, polyoxyl stearate, propylene glycol stearate, sucrose stearate, polyethylene glycol, polyethylene oxide, polypropylene oxide, polyethylene oxide-polypropylene oxide copolymers, alcohol ethoxylates, alkylphenol ethoxylates, alkyl glycosides, alkyl polyglycosides, fatty alcohols, phosphalipids, phosphatidyl chloline, phosphatidyl serine, and the like.

Other excipient components which may be included in the ophthalmic preparations are chelating agents. A useful chelating agent is edetate disodium, although other chelating agents may also be used in place or in conjunction with it.

Preservatives are used in multi-use ophthalmic compositions to prevent microbial contamination of the composition after the packaging has been opened. A number of preservatives have been developed including quaternary ammonium salts such as benzalkonium chloride; mercury compounds such as phenylmercuric acetate and thimerosal; alcohols such as chlorobutanol and benzyl alcohol; and others. In one embodiment of the invention, the preservative is benzalkonium chloride. Benzalkonium chloride is present in the invention formulations from 0.01 to 0.05% (w/v). In other embodiments the concentration is 0.015 to 0.025% (w/v). In certain embodiments, the concentration is 0.02% (w/v).

What is claimed is:

1. An aqueous ophthalmic composition comprising an S1P antagonist and at least one compound selected from the group consisting of β-blockers, adrenergic agonists, non-selective adrenergic agonists, α₂-selective adrenergic agonists, carbonic anhydrase inhibitors, cholinergic agonists, direct acting cholinergic agonists, chlolinesterase inhibitors, glutamate antagonists, Ca²⁺ channel blockers, prostamides, prostaglandins, cannabinoids, muscarinic agents, and combinations thereof.

2. The composition of claim 1 wherein the S1P antagonist is S1P2.

3. The composition of claim 1, wherein the compound is a prostamide or a prostaglandin.

4. The composition of claim 3 wherein the prostamide or prostaglandin is an EP₂ or EP₄ agonist.

5. The composition of claim 3 wherein the prostamide or prostaglandin is bimatoprost, latanoprost, or travoprost.

6. The composition of claim 1 wherein the compound is a β-blocker, an adrenergic agonist, a non-selective adrenergic agonist, or an 60 ₂-selective adrenergic agonist.

7. The composition of claim 1 wherein the compound is a muscarinic agent.

8. The composition of claim 7 wherein the muscarinic agent is pilocarpine.

9. The composition of claim 1 wherein the compound is a carbonic anhydrase inhibitor.

10. A method for lowering IOP in a subject in need thereof comprising administering to the subject a therapeutically effective amount of the composition of claim 1.

11. A method for further reducing IOP in a subject already being treated with a composition comprising at least one compound selected from the group consisting of β-blockers, adrenergic agonists, non-selective adrenergic agonists, α₂-selective adrenergic agonists, carbonic anhydrase inhibitors, cholinergic agonists, direct acting cholinergic agonists, chlolinesterase inhibitors, glutamate antagonists, Ca²⁺ channel blockers, prostamides, prostaglandins, cannabinoids, muscarinic agents, and combinations thereof;
the method comprising administering to the patient a therapeutically effective amount of the composition of claim 1.
